(19) United States
(12) Patent Application Publication
(10) Pub. No.: US 2009/0068058 A1
(43) Pub. Date: Mar. 12, 2009

(54) AIR PURIFICATION SYSTEM FOR VEHICLES AND METHOD THEREOF

(76) Inventors: Yong Chul Kim, Hwaseong-si (KR); Jae Ho Kim, Daejeon (KR)

Correspondence Address: MORGAN, LEWIS & BOCKIUS LLP (SF), One Market, Spear Street Tower, Suite 2800, San Francisco, CA, 94105, US

(21) Appl. No.: 11/972,398

(22) Filed: Jan. 10, 2008

(30) Foreign Application Priority Data
Sep. 7, 2007 (KR) 10-2007-0091125

(51) Int. Cl. A61L 9/04; G05D 99/00

(52) U.S. Cl. 422/4; 422/114

(57) ABSTRACT

Disclosed are an air purification system for a vehicle and an air purification method thereof. When an air purifier is tuned on, an aroma is discharged, and, if an air conditioner is turned on, discharge of the aroma is stopped and a deodorant is discharged for a predetermined time period. If discharge of the deodorant is stopped, the aroma is discharged again, and if the air conditioner is turned off, discharge of the aroma is stopped and the deodorant is discharged for a preset time period. If discharge of the deodorant is stopped, the aroma is discharged again.

### CROSS-REFERENCE TO RELATED APPLICATIONS

This application claims priority to Korean Application No. 10-2007-0091125, filed on Sep. 7, 2007, the disclosure of which is incorporated herein by reference.

### BACKGROUND OF THE INVENTION

1. Field of the Invention

The present invention relates to an air purification system for a vehicle and an air purification method thereof, and in particular, an air purification system for a vehicle and an air purification method thereof that can remove offensive odors generated from the air conditioner of a vehicle,.

2. Description of the Prior Art

In general, a vehicle is provided with an air cleaner and/or an aroma lamp for providing pleasant air in the cabin of the vehicle.

For example, the air cleaner receives power from a cigarette lighter socket and cleans air in the cabin of the vehicle, and the aroma lamp contains a liquid aromatic and emits a pleasant aroma into the cabin of the vehicle.

However, the air cleaner and the aroma lamp cannot remove offensive odors generated in an air conditioner when the air conditioner is turned on and off. Specifically, if the aroma from the aroma lamp is mixed with offensive odors from the air conditioner, other offensive odors are likely to be generated to cause unpleasant conditions in the cabin of the vehicle.

The information disclosed in this Background of the Invention section is only for enhancement of understanding of the background of the invention and should not be taken as an acknowledgement or any form of suggestion that this information forms the prior art that is already known to a person skilled in the art.

### SUMMARY OF THE INVENTION

Accordingly, the present invention has been made keeping in mind the above problems occurring in the prior art, and an object of the present invention is to provide an air purification system for a vehicle that discharges an aroma at normal times to provide fresh air and discharges a deodorant for a predetermined time period when an air conditioner is turned on and off to quickly remove the offensive odors generated from the air conditioner, and an air purification method thereof.

In order to achieve the above object, according to one aspect of the present invention, there is provided an air purification method for a vehicle, in which an air purifier having an aromatic module for discharging an aroma and a deodorant module for discharging a deodorant is installed in a cabin of the vehicle, separately from an air conditioner, and purifies air in the cabin of the vehicle, comprising the steps of discharging the aroma when the air purifier is turned on; stopping discharge of the aroma when the air conditioner is turned on and then discharging the deodorant for a predetermined time period; and discharging the aroma again when the discharge of the deodorant is stopped.

According to another aspect of the present invention, the air purification method further comprises the steps of stopping the discharge of the aroma when the air conditioner is turned off and then discharging the deodorant for a preset time period; and discharging the aroma again when the discharge of the deodorant is stopped.

According to another aspect of the present invention, there is provided an air purification method for a vehicle, in which an air purifier having an aromatic module for discharging an aroma and a deodorant module for discharging a deodorant is installed in a cabin of the vehicle separately from an air conditioner and purifies air in the cabin of the vehicle, comprising the steps of discharging the aroma when the air purifier is turned on in a state that the air conditioner is tuned on; discharging the deodorant using remaining electrical power of the vehicle when an engine of the vehicle is stopped; and stopping the discharge of the deodorant after a pre-selected time period has elapsed and completing the discharge of the deodorant.

According to still another aspect of the present invention, there is provided an air purification system for a vehicle comprising an air purifier installed in a cabin of the vehicle, and having an aromatic module which has an outlet port for discharging an aroma, a deodorant module which has an outlet port for discharging a deodorant and a solenoid actuator, which opens and closes the outlet ports to selectively discharge the aroma or the deodorant; and a control unit for controlling the air purifier in a manner such that, when the air purifier is turned on, the aroma is discharged, and, if an air conditioner is turned on, discharge of the aroma is stopped and then the deodorant is discharged for a predetermined time period, such that, if discharge of the deodorant is stopped, the aroma is discharged again, and if the air conditioner is turned off, discharge of the aroma is stopped and then the deodorant is discharged for a preset time period, and such that, if discharge of the deodorant is stopped, the aroma is discharged again.

According to a still further aspect of the present invention, there is provided an air purification system for a vehicle comprising an air purifier installed in a cabin of the vehicle, and having an aromatic module which has an outlet port for discharging an aroma, a deodorant module which has an outlet port for discharging a deodorant and a solenoid actuator which opens and closes the outlet ports to selectively discharge the aroma or the deodorant; and a control unit for controlling the air purifier in a manner such that, with an air conditioner is on, if the air purifier is turned on, the aroma is discharged, and if an engine is stopped, the deodorant is discharged using remaining electrical power of the vehicle, and that, when the discharge of the deodorant is completed, that is, a pre-selected time period has elapsed, the discharge of the deodorant is stopped.

### BRIEF DESCRIPTION OF THE DRAWINGS

The above and other features of the present invention will now be described in detail with reference to certain exemplary embodiments thereof illustrated the accompanying drawings which are given hereinbelow by way of illustration, and thus are not limitative of the present invention, and wherein:

FIG. 1 is a block diagram illustrating an air purification method for a vehicle in accordance with an exemplary embodiment of the present invention;

FIG. 2 is a block diagram illustrating an air purification method for a vehicle in accordance with another exemplary embodiment of the present invention;

FIG. 3 is a structural view illustrating an air purification system for a vehicle in accordance with still another exemplary embodiment of the present invention; and

FIG. 4 is a view illustrating a vehicle in which the air purification system for a vehicle according to the present invention is installed.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION OF THE INVENTION

Reference will now be made in detail to a preferred embodiment of the present invention, an example of which is illustrated in the accompanying drawings and described below. While the invention will be described in conjunction with an exemplary embodiment, it should be understood that the description is not intended to limit the invention to the exemplary embodiment. On the contrary, the invention is intended to cover not only the exemplary embodiment, but also various alterative, modifications, equivalents and other embodiments, which maybe included within the spirit and scope of the invention as defined by the appended claims.

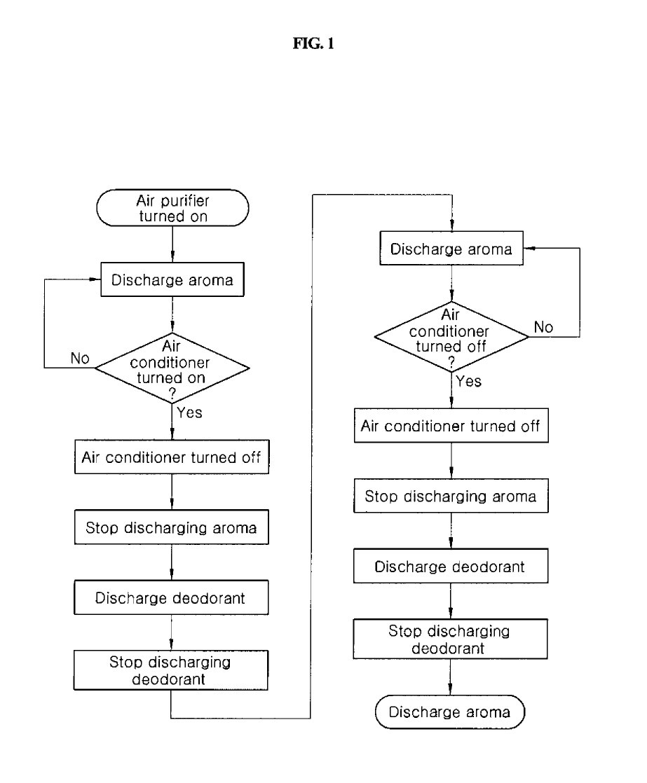

FIG. 1 is a block diagram illustrating an air purification method for a vehicle in accordance with an exemplary embodiment of the present invention;

Referring to FIG. 1, in an air purification method for a vehicle in accordance with an exemplary embodiment of the present invention, an air purifier, which has an aromatic module for discharging an aroma and a deodorant module for discharging a deodorant, is employed. In the air purification method in accordance with an exemplary embodiment of the present invention, if the air purifier of the vehicle is turned on, the aroma is discharged, and, if an air conditioner is turned on, the deodorant is discharged for a predetermined time period. Also, if the air conditioner is turned off, the discharge of the aroma is stopped, and the deodorant is discharged from the air purifier for a predetermined time period. In this way, the offensive odors generated from the air conditioner can be removed.

Concretely speaking, if the air purifier of the vehicle is turned on, the aroma is discharged from the air purifier. The aroma discharged from the air purifier creates pleasant conditions in the cabin of the vehicle, so that an occupant can enjoy a fragrant smell.

If the air conditioner is turned on, discharge of the aroma is stopped, and the deodorant is discharged for the predetermined time period to remove the offensive odors generated from the air conditioner. The reason for is that, if the aroma is continuously discharged after the air conditioner is initially turned on, the aroma and the offensive odors generated from the air conditioner may be mixed with each other, and may produce other offensive odors.

If sufficient time elapses so oat the offensive odors generated from the air conditioner are removed, the discharge of the deodorant is stopped. The time for discharging the deodorant can be changed depending upon the air conditioning conditions in the cabin of the vehicle. If the offensive odors in the cabin are not serious, because the air conditioner is used for a short period, the time to discharge the deodorant can be decreased. If the offensive odors in the cabin are serious, since the air conditioner is used for a long period, the time to discharge the deodorant can be lengthened.

After discharge of the deodorant is stopped, the aroma is discharged again, so that the fragrant smell can be provided into the cabin of the vehicle.

If the air conditioner is turned off, in order to remove the offensive odors generated due to the turning-off of the air conditioner, the discharge of aroma is stopped, and the deodorant is discharged from the air purifier for the preset time period. If discharge of the deodorant is completed, the aroma is discharged again, so that the fragrant smell can be provided again into the cabin of the vehicle.

An odor level indicator for sensing the level of the offensive odors in the cabin of the vehicle can be separately installed in the cabin of the vehicle. As the level of the offensive odors in the cabin of the vehicle is sensed by the odor level indicator, the time for which to discharge the deodorant can be adjusted depending upon the level of the offensive odors.

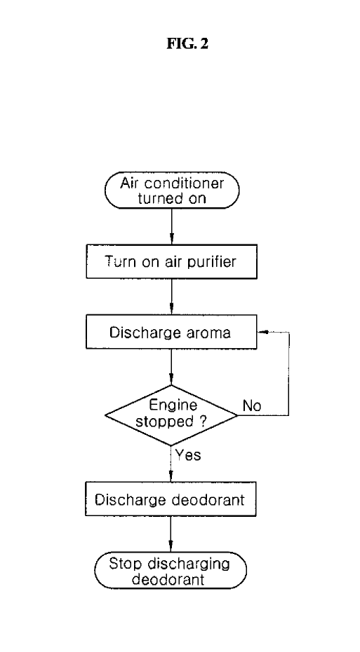

FIG. 2 is a block diagram illustrating an air purification method for a vehicle in accordance with another exemplary embodiment of the present invention;

Referring to FIG. 2, in the air purification method in accordance with another exemplary embodiment of the present invention, in a state in which the air conditioner of the vehicle is turned on, the aroma is discharged from the air purifier, and then, if the engine of the vehicle is stopped, the deodorant is discharged from the air purifier. In this way, the offensive odors generated from the air conditioner can be removed.

Concretely speaking, with the air conditioner of the vehicle turned on, the air purifier is turned on and discharges the aroma into the cabin of the vehicle.

When the engine of the vehicle is stopped in a state that the air conditioner is turned on, the deodorant is discharged from the air purifier using the electrical power remaining in the vehicle. Some remaining amount of electrical power that is applied for a few seconds to fold the outside rearview mirror and/or close an opened door window when the engine is stopped can be used.

When the discharge of the deodorant is completed, that is, when a pre-selected time period has elapsed, the discharge of the deodorant is stopped. A few seconds is a sufficient time to discharge the deodorant.

In this way, the offensive odors generated from the air conditioner can be removed by the deodorant that is discharged after the air conditioner is turned off.

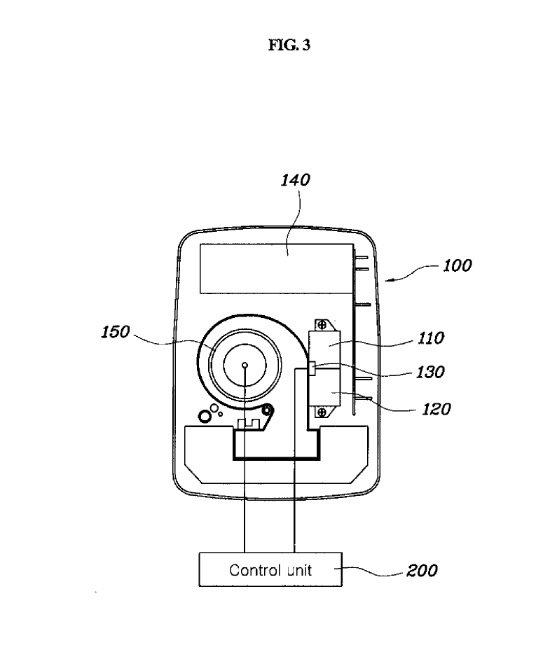

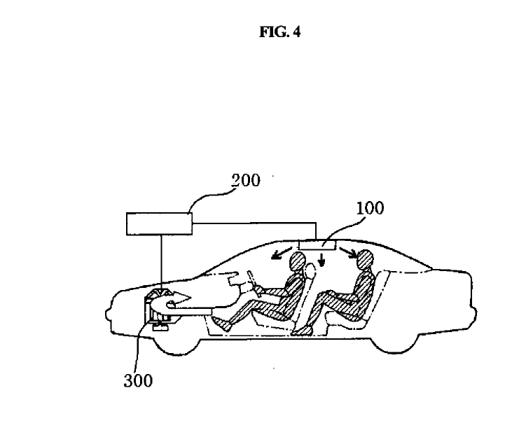

FIG. 3 is a structural view illustrating an air purification system for a vehicle in accordance with still another exemplary embodiment of the present invention and FIG. 4 is a view illustrating a vehicle in which the air purification system for a vehicle according to the present invention is installed.

Referring to FIGS. 3 and 4, an air purification system in accordance with still another exemplary embodiment of the present invention includes an air purifier 100, which is installed on the roof panel of the vehicle, and a control unit 200. The air purifier 100 has an aromatic module 110, a deodorant module 120, and a solenoid actuator 130 for selectively discharging the aroma and the deodorant. The control unit 200 controls the operation of the air purifier 100 in a manner such that, when the air purifier 100 is turned on, the aroma is discharged, and when an air conditioner 300 is turned on and off, the deodorant is discharged.

The air purifier 100 includes the aromatic module 110, which has an outlet port for discharging the aroma, the deodorant module 120, which has an outlet port for discharging the deodorant, the solenoid actuator 130, for selectively opening and closing the outlet port of the aromatic module 110 and the outlet port of the deodorant module 120 to selectively discharge the aroma and the deodorant from the aromatic module 110 and the deodorant module 120, a fan 150, for directing the aroma or the deodorant into the cabin of the vehicle, and a filter 140 for filtering out the pollutants present in the cabin.

The control unit 200 for controlling the operations of these component parts can be provided in the air purifier 100, or can be designed as a separate part which is electrically connected with the air purifier 100. Moreover, the control unit 200 can have a signal reception section, which can receive an air conditioner operation signal from the controller of an air conditioning system.

The control unit 200 according to one exemplary embodiment of the present invention can perform a control function as described below. When the air purifier 100 of the vehicle is tuned on, the aroma is discharged from the air purifier 100, and if the air conditioner 300 is turned on, the discharge of the aroma is stopped and the deodorant is discharged for the predetermined time period from the air purifier 100. If the discharge of the deodorant is stopped, the aroma is discharged again, and if the air conditioner 300 is turned off, the discharge of the aroma is stopped and the deodorant is discharged for the preset time period from the air purifier 100. Then, if the discharge of the deodorant is stopped, the aroma can be discharged again into the cabin of the vehicle.

Further, the control unit 200 according to another exemplary embodiment of the present invention can perform a control function as described below. In a state in which the air conditioner 300 of the vehicle is turned on, if the air purifier 100 is turned on, the aroma is discharged, and then, if the engine is stopped, the deodorant is discharged using the power remaining in the vehicle. If the discharge of the deodorant is completed with the lapse of the pre-selected time period, the discharge of the deodorant is stopped.

In this way, the offensive odors generated in the air conditioner 300 and introduced into the cabin of the vehicle can be removed by the deodorant which is discharged after the air conditioner 300 is tuned off.

As is apparent from the above description, an exemplary embodiment of the present invention provides advantages in that a deodorant is discharged for a predetermined time period under a condition in which an air conditioning system of a vehicle emits a strong smell, that is, after an air conditioner is turned on and off, to quickly remove the offensive odors generated in the air conditioner and maintain pleasant conditions in the cabin of the vehicle.

Also, in another exemplary embodiment of the present invention, when an engine is stopped while the air conditioner is operated, the offensive odors generated from the air conditioner can be removed immediately after the engine is stopped, so that a driver can start the vehicle again under pleasant conditions.

Although preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. An air purification method for a vehicle, in which an air purifier, having an aromatic module for discharging an aroma and a deodorant module for discharging a deodorant, is installed in a cabin of the vehicle separately from an air conditioner and purifies air in the cabin of the vehicle, comprising the steps of:
discharging the aroma when the air purifier is turned on;
stopping discharge of the aroma when the air conditioner is turned on and discharging the deodorant for a predetermined time period; and
discharging the aroma again when discharge of the deodorant is stopped.

2. The air purification method according to claim 1, further comprising the steps of:
stopping discharge of the aroma when the air conditioner is turned off and then discharging the deodorant for a preset time period; and
discharging the aroma again when discharge of the deodorant is stopped.

3. An air purification method for a vehicle, in which an air purifier, having an aromatic module for discharging an aroma and a deodorant module for discharging a deodorant, is installed in a cabin of the vehicle separately from an air conditioner, and purifies air in the cabin of the vehicle, comprising the steps of:
discharging the aroma when the air purifier is turned on in a state that the air conditioner is turned on;
discharging the deodorant using remaining electrical power of the vehicle when an engine of the vehicle is stopped; and
stopping discharge of the deodorant after a lapse of a preselected time period.

4. An air purification system for a vehicle comprising:
an air purifier installed in a cabin of the vehicle, and having an aromatic module which has an outlet port for discharging an aroma, a deodorant module which has an outlet port for discharging a deodorant and a solenoid actuator which opens and closes the outlet ports to selectively discharge the aroma or the deodorant; and
a control unit for controlling the air purifier in a manner such that, when the air purifier is tuned on, the aroma is discharged, and, when an air conditioner is turned on, discharge of the aroma is stopped and then the deodorant is discharged for a predetermined time period, such that, if discharge of the deodorant is stopped, the aroma is discharged again, and if the air conditioner is turned off, discharge of the aroma is stopped and then the deodorant is discharged for a preset time period, and such that, if discharge of the deodorant is stopped, the aroma is discharged again.

5. An air purification system for a vehicle comprising:
an air purifier installed in a cabin of the vehicle, and having an aromatic module which has an outlet port for discharging an aroma, a deodorant module which has an outlet port for discharging a deodorant and a solenoid actuator which opens and closes the outlet ports to selectively discharge the aroma or the deodorant; and
a control unit for controlling the air purifier in a manner such that, with an air conditioner turned on, if the air purifier is turned on, the aroma is discharged, and if an engine is stopped, the deodorant is discharged using remaining electric power of the vehicle, and that, if discharge of the deodorant is completed upon a lapse of a pre-selected time period, discharge of the deodorant is stopped.
